(19) United States
(12) Patent Application Publication
(10) Pub. No.: US 2026/0137921 A1
(43) Pub. Date: May 21, 2026

(54) PHARMACEUTICAL CONNECTION APPARATUS FOR A HOSE LINE

(71) Applicant: Bausch + Stroebel SE + Co. KG, Ilshofen (DE)

(72) Inventors: Dominik Kronmueller, Ilshofen (DE); Robin Hofmann, Ilshofen (DE); Alexandra Bisswang, Ilshofen (DE)

(21) Appl. No.: 19/452,376

(22) Filed: Jan. 18, 2026

Related U.S. Application Data
(63) Continuation of application No. PCT/EP2024/069569, filed on Jul. 10, 2024.

(30) Foreign Application Priority Data
Jul. 19, 2023 (DE) 10 2023 119 099.7

(51) Int. Cl. A61M 39/10; A61M 39/00

(52) U.S. Cl. CPC A61M 39/10; A61M 2039/0009; A61M 2039/1033

(57) ABSTRACT

A pharmaceutical connection apparatus for a hose line includes a base body. An insertion region for the hose line and a channel are formed in the base body. The insertion region opens into the channel, and a contact region for the hose line is arranged on the entry side of the channel. A clamping body, through which the hose line is guided, has or forms a clamping region facing the base body. A clamping receptacle of the apparatus has a conical taper. A fixing device of the apparatus fixes the clamping body relative to the base body in a usage state. In the usage state, the clamping body engages into the clamping receptacle, and the clamping region applies a clamping force to the hose line. The hose line extends through the insertion region and rests with an end face on the contact region, which forms a sealing seat.

### CROSS-REFERENCE TO RELATED APPLICATIONS

This application is a continuation of international application number PCT/EP2024/069569, filed on Jul. 10, 2024, and claims the benefit of German application number 10 2023 119 099.7, filed on Jul. 19, 2023, both of which are incorporated herein by reference in their entireties and for all purposes.

### FIELD

The present disclosure relates to a pharmaceutical The pharmaceutical connection apparatus for a flexible hose line, comprising a base body in which a channel is formed which is in flow connection with the hose line. The pharmaceutical connection apparatus may, for example, comprise a pharmaceutical filling needle for filling a container, it being possible for the filling needle to be comprised or formed by the base body.

### BACKGROUND

Such a The pharmaceutical connection apparatus is used, for example, in a facility for processing pharmaceutical containers. The containers may, for example, be vials, syringes, cartridges, or ampoules. For example, use of the pharmaceutical connection apparatus for connecting the flexible hose line to a filling needle in a filling station is conceivable. The, for example liquid, product to be filled into the containers passes through the hose line and the filling needle into the container.

However, the present disclosure is not limited to this. It is conceivable that the pharmaceutical connection apparatus could be used to connect the hose line to an object that differs from a filling needle.

It is known to use hose lines made of PTFE (polytetrafluoroethylene) that have a pharmaceutical-grade design. However, it is necessary to clean the hose lines after production cycles. This can be done, for example, by disassembling the pharmaceutical connection apparatuses and cleaning the hose lines externally. However, cleaning within the system (in-line) is desirable, without having to disassemble the pharmaceutical connection apparatus for this purpose. Such a process is referred to, for example, as CIP/SIP (CIP, Cleaning in Place/SIP, Sterilization in Place). In in-line sterilization, the hose line is subjected, for example, to a flow of hot steam, which has a temperature of approximately 135° C. and a pressure of approximately 2 bar and above. In this case, PTFE hose lines can deform, which can impair the tightness when connected to the pharmaceutical connection apparatus.

The pharmaceutical connection apparatuses are known in which the hose line must be slipped over a connecting nipple of the base body. This requires a lot of maintenance. Repeated cleaning or sterilization can also lead to leaks due to deformation of the hose line at the end portion that is slipped over the base body.

An object underlying the present disclosure is to provide a The pharmaceutical connection apparatus for a flexible hose line, the pharmaceutical connection apparatus having a structurally simple design and preferably being easy to handle.

### SUMMARY

In a first aspect of the disclosure, a pharmaceutical The pharmaceutical connection apparatus for a flexible hose line is provided. The pharmaceutical connection apparatus comprises a base body in which an insertion region for the hose line and a channel are formed, the insertion region opening into the channel and on the entry side of the channel a contact region for the hose line being arranged. The pharmaceutical connection apparatus further comprises a clamping body, through which the hose line is guided and which comprises or forms a clamping region facing the base body. Further, the pharmaceutical connection apparatus comprises a clamping receptacle, which has a, for example, conical taper in the direction of the contact region, as well as a fixing device for fixing the clamping body relative to the base body in a usage state of the pharmaceutical connection apparatus. In the usage state, the clamping body engages, by means of the clamping region, into the tapered clamping receptacle, and the clamping region applies a clamping force to the hose line radially from outside. The hose line extends by means of an end portion through the insertion region and rests with an end face on the contact region, which forms a sealing seat.

### BRIEF DESCRIPTION OF THE DRAWINGS

The foregoing summary and the following description may be better understood in conjunction with the drawing figures, of which:

FIG. 1: is a perspective view of the pharmaceutical connection apparatus in accordance with the present disclosure in a preferred embodiment;

FIG. 2: is a perspective exploded view (in part) of the pharmaceutical connection apparatus from FIG. 1;

FIG. 3: is a longitudinal sectional view (in part) of the pharmaceutical connection apparatus from FIG. 1;

FIGS. 4 and 5: are sectional views (partial views), corresponding to FIG. 3, of further preferred embodiments of the pharmaceutical connection apparatus in accordance with the present disclosure;

FIG. 6: is a perspective view of the pharmaceutical connection apparatus in accordance with the present disclosure in a further preferred embodiment;

FIG. 7: is a perspective exploded view of the pharmaceutical connection apparatus from FIG. 6 (in part);

FIG. 8: is a longitudinal sectional view of the pharmaceutical connection apparatus from FIG. 6 (partial view);

FIG. 9: is a view of detail A in FIG. 8 in a further preferred embodiment;

FIG. 10: is a perspective view of the pharmaceutical connection apparatus in accordance with the present disclosure in a further preferred embodiment;

FIG. 11: is a perspective exploded view of the pharmaceutical connection apparatus from FIG. 10 (in part);

FIG. 12: is a longitudinal sectional view (in part) of the pharmaceutical connection apparatus from FIG. 10;

FIG. 13: is a perspective exploded view (in part) of a further embodiment of the pharmaceutical connection apparatus in accordance with the present disclosure; and

FIG. 14: is a longitudinal sectional view of the pharmaceutical connection apparatus from FIG. 13 (in part).

### DETAILED DESCRIPTION

Although the present disclosure is illustrated and described herein with reference to specific embodiments, the present disclosure is not intended to be limited to the details shown. Rather, various modifications may be made in the details within the scope and range of equivalents and without departing from the present disclosure.

The present disclosure relates to a pharmaceutical The pharmaceutical connection apparatus for a flexible hose line, comprising
a base body, in which an insertion region for the hose line and a channel are formed, wherein the insertion region opens into the channel and on the entry side of the channel a contact region for the hose line is arranged;
a clamping body, through which the hose line is guided and which comprises or forms a clamping region facing the base body;
a clamping receptacle, which has an in particular conical taper in the direction of the contact region; and
a fixing device for fixing the clamping body relative to the base body in a usage state of the pharmaceutical connection apparatus;
wherein in the usage state, the clamping body engages, by means of the clamping region, into the tapered clamping receptacle and the clamping region applies a clamping force to the hose line radially from outside, and wherein the hose line extends by means of an end portion through the insertion region and rests with an end face on the contact region, which forms a sealing seat.

In the pharmaceutical connection apparatus in accordance with the present disclosure, a flow connection from the hose line to the channel can be achieved by the hose line resting against the end face of the contact region on the entry side of the channel. The contact region forms a corresponding sealing seat for the end face of the hose line. The hose line extends with an end portion through the insertion region in the base body, which opens into the channel. In particular, it is provided here that the cross-sectional area of the insertion region, in which the end portion of the hose line is arranged, is larger than the cross-sectional area of the channel. A clamping body is provided which, when the pharmaceutical connection apparatus is used as intended, is arranged in the clamping receptacle of the pharmaceutical connection apparatus. The hose line is, for example, guided through a through-opening in the clamping body. The hose line is subjected via the clamping body to a force radially from the outside, and can thus be clamped. The fixing device makes it possible to fix the clamping body relative to the base body in the usage state. This allows the hose line to be held immovably relative to the base body, so that a sufficient seal can be achieved at the sealing seat on the base body. This seal is preferably suitable for cleaning and/or sterilization without disassembling the pharmaceutical connection apparatus, especially for CIP/SIP applications. In practice, the pharmaceutical connection apparatus proves to be easy to handle. Preferably, manual and in particular tool-free assembly and/or disassembly of the pharmaceutical connection apparatus is conceivable here.

It may be provided, for example, that the clamping body is elastically deformable, at least in portions. This is discussed below.

The clamping body can, for example, be made of a plastics material. For example, production from PEEK (polyetheretherketone) is conceivable.

The base body and/or a fixing body of the fixing device can, for example, be made of a plastics material or a metal material.

Preferably, the hose line is free from interference by the base body. In the present case, this can be understood, for example, to mean that the hose line is not slipped over the base body. In a manner deviating from a conventional The pharmaceutical connection apparatus, in the preferred embodiment of the pharmaceutical connection apparatus in accordance with the present disclosure, the need for the base body to engage with the hose line via a connecting nipple or the like is eliminated. Rather, it is favorably sufficient for the hose line to simply rest on the contact region at the end face, it being able to be gripped radially on the outside by the base body.

In a preferred embodiment of the present disclosure, the base body forms the clamping receptacle, so that the clamping region is held in a clamping fit on the base body in the usage state, for applying the clamping force to the hose line. In terms of design, the clamping receptacle with the taper is, for example, located at the insertion region of the base body or formed by it. The clamping body engages with the clamping region in the base body and is held there via the clamping fit for applying the clamping force to the hose line. Such an embodiment in particular has a structurally simple design.

The fixing device of the last-mentioned advantageous embodiment comprises, for example, cooperating fixing elements on the clamping body and on the base body, the clamping body being directly connected to the base body. This allows for a structurally simple design, it being possible for the fixing device to be formed by the clamping body and the base body, without a separate fixing body being used.

The fixing elements can be or comprise, for example, screw elements, clamping elements and/or locking elements. The use of screw elements can be advantageous. In this case, the clamping force exerted on the hose line across the clamping region and/or the force on the hose line at the sealing seat can preferably be influenced by the depth of the screw connection between the clamping body and the base body.

The clamping body preferably engages in the base body, an external thread of the clamping body being screwed to an internal thread of the base body. Alternatively, for example, the clamping body can overlap the base body at least in portions, an internal thread of the clamping body being screwed to an external thread of the base body.

In a preferred embodiment of the present disclosure, it can be provided that the fixing device comprises a fixing body formed separately from the clamping body, which is connected to the base body via fixing elements, the clamping body being supported directly or indirectly on the fixing body with a side facing away from the base body. The clamping body can be subjected to a force via the fixing body, which force is directed towards the base body. This allows for reliable fixation of the clamping element to be achieved.

The fixing elements can be or comprise screw elements, clamping elements and/or locking elements, as described above. Screw elements can be advantageous for the reasons already explained. It may be provided that, in particular, the fixing body overlaps the base body and an internal thread of the fixing body is screwed to an external thread of the base body.

The fixing body can be constructed in a simple way, for example in the form of a cap nut that overlaps the clamping body and overlaps the base body at least in portions and is connected thereto.

In the advantageous embodiments of the present disclosure described above, the clamping receptacle is formed by the base body. Alternative embodiments may provide that the clamping receptacle is not formed by the base body, but by a separate receiving body.

For example, it may be expedient if the pharmaceutical connection apparatus comprises a receiving body that is configured separately from the base body and that forms the clamping receptacle, preferably the receiving body and the base body bearing on each other over mutually facing end faces in the usage state of the pharmaceutical connection apparatus. In this embodiment, the receiving body forms the clamping receptacle into which the clamping body engages. In the usage state, the clamping body can be fixed via the fixing device, which can be in operative connection with the receiving body and/or with the clamping body, in order to fix the hose line. The end portion of the hose line is arranged in portions outside the base body, for example at the clamping region, and can engage in the insertion region on the base body.

It is favorable in particular if the receiving body and the clamping body are connected to each other.

In terms of design, the receiving body can, for example, be configured as a sleeve that surrounds the clamping body at least in portions. For example, the clamping body extends through or engages in the sleeve. The clamping body itself can, for example, be configured in the shape of a sleeve.

The connection between the receiving body and the clamping body can be made, for example, by screwing, clamping and/or locking. For example, a screw connection can be advantageous. This allows, for example, the adjustment of the clamping force exerted on the hose line and/or the force applied to the end face of the hose line.

It is conceivable that an internal thread of the receiving body is screwed to an external thread of the clamping body.

It may be provided that the receiving body forms a fixing body, so that the fixing device does not comprise a further, separate fixing body.

In another advantageous embodiment, it is provided that the fixing device comprises a fixing body formed separately from the receiving body and/or the clamping body, which is connected to the base body via fixing elements, preferably the receiving body and/or the clamping body being supported directly or indirectly on the fixing body with a side facing away from the base body. The fixing body can be used to fix the receiving body and/or the clamping body in the usage state. Favorably, the receiving body and/or the clamping body is supported directly or indirectly on the fixing body. When the receiving body and clamping body are connected, it may be sufficient if one of these two bodies is supported directly or indirectly on the fixing body.

The fixing elements can be, for example, screw elements, clamping elements and/or locking elements. For example, screw connections using screw elements are advantageous for the reasons already explained.

The fixing body can, for example, overlap the receiving body and the base body at least in portions, an internal thread of the fixing body being screwed to an external thread of the base body.

The fixing body can be configured in a structurally simple way, for example as a cap nut.

It can prove to be favorable if the pharmaceutical connection apparatus comprises a biasing element that is effective between the receiving body and/or the clamping body on the one hand, and the fixing body on the other, whereby the receiving body and/or the clamping body can be applied with a force directed towards the base body, in particular towards the contact region. The biasing force can act in particular on the clamping body, for example directly or indirectly, if the receiving body is connected to the clamping body as explained above. The biasing force can be used to improve the sealing of the hose line at the sealing seat. In particular, this embodiment may prove favorable for a CIP/SIP application for cleaning and/or sterilizing the hose line in the assembled state. In practice, it is found that a long-term seal at the sealing seat can also be achieved if multiple hot steam sterilizations are carried out at a temperature of, for example, approximately 135° C. and a pressure of approximately 2 bar and above (for example, approximately 2.2 bar).

The biasing element is configured, for example, as a spring element, in particular as a coil spring.

In order to achieve a structurally simple design, it is favorable if the biasing element is integrally formed (monolithically) with the receiving body or integrally formed (monolithically) with the clamping body.

For example, an additive manufacturing process is used for one-piece production. For example, the biasing element is produced using 3D printing, together with the receiving body or together with the clamping body.

A compact design can preferably be achieved in that the biasing element is arranged radially on the inside with respect to the fixing body and/or in that the biasing element surrounds the clamping body and/or the receiving body.

The clamping body can, for example, be configured as an elongate sleeve.

A possible embodiment of the clamping region is discussed in more detail in the following.

The clamping body can advantageously comprise two or more clamping elements on the clamping region, which clamping elements are preferably elastically deformable and separated from each other by a respective gap, and which are held at a common connection region. The clamping elements, which may be shaped like fingers, can protrude from the connection region. The clamping elements are separate from each other and can preferably be deformed independently of each other, preferably a spring-like design being conceivable. In the usage state, the clamping elements at the clamping region are subjected to the force directed towards the hose line, so that the force is distributed at several points around the periphery of the hose line.

For example, more than two clamping elements and respective gaps between them may be provided.

In an advantageous embodiment of the present disclosure, for example four clamping elements can be provided, which each have an angular distance of 90° from each other in the peripheral direction of the hose line.

It is conceivable that two clamping elements are provided, positioned opposite each other.

Alternatively or additionally, two gaps can be provided, which are positioned opposite each other.

The two or more clamping elements are advantageously configured identically.

The angular distance between respective adjacent clamping elements is preferably identical.

It can prove favorable if the clamping elements comprise radial inner projections, which are preferably configured in a toothed shape. In practice, it is found that this allows an axial force to be exerted on the hose line in the direction of the base body. This allows for an improved seal at the sealing seat.

The clamping elements have, for example, a sawtooth shape in cross-section facing the hose line.

The base body can be configured to be tapered at least in portions at the insertion region, the taper in particular being conical. This can apply, for example, if the clamping receptacle is not formed by the base body.

The contact region can, for example, define a contact plane oriented perpendicularly to the channel. For example, a shoulder is arranged on the entry side of the channel, against which shoulder the end face of the hose line rests.

In a manner deviating from the above embodiment with a contact plane oriented perpendicularly to the channel, in an advantageous embodiment the contact region can, for example, be configured as a conical taper (e.g. 45° taper) of the base body in the direction of the channel. This offers the possibility of compressing the hose line in order to achieve a good seal at the sealing seat.

The contact region can be located at the end of the taper of the clamping region. Alternatively, a cylindrical portion of the insertion region can be formed on the base body. The cylindrical portion for example encloses the hose line, in particular in a positive-locking manner.

The base body may comprise or form a pharmaceutical filling needle for filling pharmaceutical containers. Alternatively, the pharmaceutical connection apparatus can comprise a filling needle that is connected or connectible to the base body.

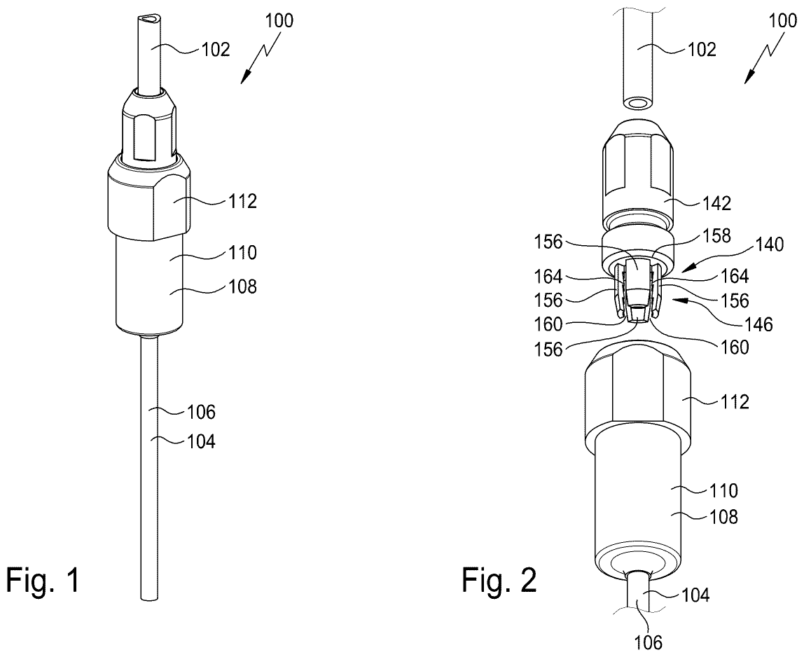

A plurality of advantageous embodiments of the pharmaceutical connection apparatus in accordance with the present disclosure are described below with reference to the drawing, beginning with an advantageous embodiment of the pharmaceutical connection apparatus designated with reference numeral 100, which is shown in FIG. 1 to 3.

Identical reference numerals are used for identical or like-acting features and components of the various embodiments. Following the explanation of the first embodiment 100, only the essential differences of the other embodiments are described. The properties, advantages and effects described in connection with the first embodiment apply accordingly to the further embodiments, even if this is not explained in detail.

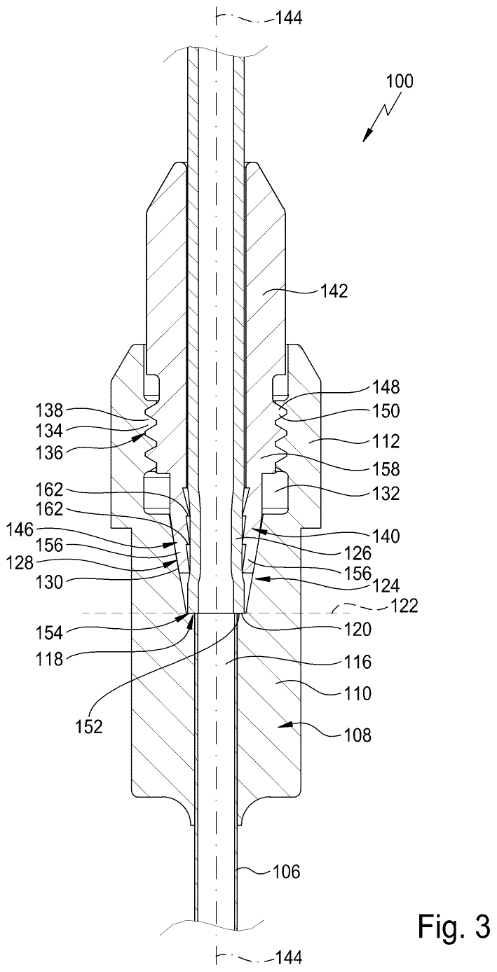

FIG. 1 shows the pharmaceutical connection apparatus 100 in a perspective view, FIG. 2 in a perspective exploded view. FIG. 3 is a sectional view of the pharmaceutical connection apparatus 100.

The pharmaceutical connection apparatus 100 is used to connect a flexible hose line 102 to an object 104. In the present exemplary embodiment, the object 104 is a filling needle 106, which is used in a facility for filling pharmaceutical containers. The facility and the containers are not shown in the drawing itself.

The pharmaceutical connection apparatus 100 is included together with the filling needle 106 in a filling station of the facility. A product to be filled, in particular a liquid, is supplied via the hose line 102. The container is filled via the filling needle 106 which engages in the container.

This application of the present disclosure is by way of example and non-limiting. It may be provided that instead of connecting the hose line 102 to the filling needle 106, a different type of connection is made with an object 104 that differs from the filling needle 106.

As mentioned, the hose line 102 is flexible and consists of a pharmaceutical-grade material, such as PTFE (polytetrafluoroethylene).

The pharmaceutical connection apparatus 100 comprises a base body 108. In the present case, the base body 108 forms the filling needle 106, or the filling needle 106 is connected to the base body 108.

In the present case, the base body 108 is substantially cylindrical in design, comprising a first portion 110 and a second portion 112, which are aligned coaxially with respect to an axis 114 of the base body 108. The diameter of the second portion 112 is larger than that of the first portion 110. However, this is only one embodiment by way of example.

In the base body 108 a channel 116 is formed which has the axis 114 and comprises a contact region 118 on the entry side, i.e. in the direction of flow of the liquid. The channel 116 is in flow connection with the hose line 102.

The contact region 118 is formed by a shoulder 120, which defines a contact plane 122. The contact plane 122 is oriented transversely and in the present case in particular perpendicularly to the axis 114.

Furthermore, the base body 108 comprises an insertion region 124 in which, in the present example, the hose line 102 extends with an end portion 126. The insertion region 124 opens into the channel 116 and is arranged immediately in front of the channel 116 in the direction of the inserted hose line 102.

The pharmaceutical connection apparatus 100 comprises a clamping receptacle 128. The clamping receptacle 128 is formed in the present case by the base body 108 at the insertion region 124. The clamping receptacle 128 has a taper 130, which in the present example is in particular conical in design. Overall, the clamping receptacle 128 tapers in the direction of the contact region 118.

On the entry side of the base body 108, a receiving space 132 is formed, in the present case at the second portion 112. At the receiving space 132, the base body 108 has a fixing element 134, which is part of a fixing device 136 of the pharmaceutical connection apparatus 100. The fixing element 134 is configured in the present case as a screw element 138 with an internal thread of the base body 108.

The pharmaceutical connection apparatus 100 comprises a clamping body 140. The clamping body 140 is configured in the present case as an elongate sleeve 142. The clamping body 140 defines an axis 144 which coincides with the axis 114 in the intended usage state of the pharmaceutical connection apparatus 100. In the usage state, the clamping body 140 is connected to the base body 108 for the adaptation of the hose line 102.

In the present case, the hose line 102 passes through the clamping body 140 at a central through-opening.

On the side facing the contact region 118, the clamping body 140 comprises a clamping region 146. In the present case, the hose line 102 extends beyond the clamping region 146 and over the clamping Body 140.

As a further fixing element 148 of the fixing device 136, a screw element 150 is provided on the clamping body 140, formed by an external thread. The fixing elements 148, 134, can cooperate for fixing the clamping body 140 to the base body 108.

In the present case, the clamping body 140 is dimensioned such that it engages in a form-fitting manner with the base body 108. The screw elements 138, 150 are screwed together so that the clamping body 140 is reliably held on the base body 108 in the usage state.

The clamping region 146 engages in the tapered clamping receptacle 128. This ensures that the clamping region 146 is held in a clamping fit on the base body 108. This allows the hose line 102 to be subjected to a clamping force radially from the outside (in the present case with respect to the axis 144, which coincides with the axis of the hose line 102). As a result, the hose line 102 is reliably held on the clamping body 140 and thereby adapted to the filling needle 106 by means of the pharmaceutical connection apparatus 100.

The end face 152 of the hose line 102 rests against the contact region 118 which forms a sealing seat 154. In this case, the hose line 102 abuts against the shoulder 120. This is sufficient in the present case to achieve an adequate seal between the hose line 102 and the base body 108.

It is in particular not necessary for the base body 108 to engage in the hose line 102 as with conventional The pharmaceutical connection apparatuses.

The pharmaceutical connection apparatus 100 also proves to be structurally simple in its manufacture and easy to handle. For example, manual and in particular tool-free assembly and/or disassembly is conceivable.

As can be seen in particular from FIGS. 2 and 3, the clamping body 140 comprises a plurality of clamping elements 156 at the clamping region 146. The clamping elements 156 extend from a common connection region 158 of the sleeve 142 in the direction of the contact region 118. In the clamping receptacle 128, the clamping elements 156 cooperate in particular with the base body 108.

The clamping elements 156 are made of an elastically deformable material. For example, production from a plastics material, such as PEEK, is conceivable. The clamping body 140 can be made entirely from the plastics material and can, for example, be integrally formed.

Four clamping elements 156 are provided in the present case, the number of which is merely by way of example. A gap 160 is arranged between neighboring clamping elements 156. In each case two clamping elements 156 are positioned opposite each other. Similarly, in each case two gaps 160 are positioned opposite each other.

The clamping elements 156 each extend over the same angular range with respect to the axis 144 and have identical angular distances from each other.

In particular, the clamping elements 156 are configured identically.

On the radially inner side facing the hose line 102, the clamping elements 156 have projections 162, preferably a plurality of projections 162 in each case. The projections 162 are toothed, and in the present example are configured such that the clamping element 156 has a sawtooth shape in the cross-section facing the hose line 102 (FIG. 3).

In the usage state, the clamping elements 156 are pressed together by the clamping receptacle 128. In this case, the projections 162 engage with the hose line 102. This serves not only to reliably fix the hose line 102, but also to ensure that the hose line 102 is subjected to an axial force when screwed together, thereby achieving a reliable seal at the contact region 118.

Depending on the depth of the screw connection of the clamping body 140 with the base body 108, the force application on the hose line 102 can be influenced, and thus also the force between the hose line 102 and the contact region 118.

For cleaning purposes, in particular for sterilization purposes after a production cycle, the pharmaceutical connection apparatus 100 can be disassembled for ease of handling. Even after repeated sterilization using hot steam, the hose line 102 remains in such a shape that repeated use and a fluid-tight connection using the pharmaceutical connection apparatus 100 are possible.

Depending on the dimensions of the hose line 102, the pharmaceutical connection apparatus in accordance with the present disclosure can have different dimensions. In practice, for example, it is common to use hoses with an inside diameter of approximately 0.9 mm upwards to approximately 7.5 mm, and in some cases even larger. For example, a wall arrangement of the hose line 102 is approximately 0.8 mm to 1.5 mm thick.

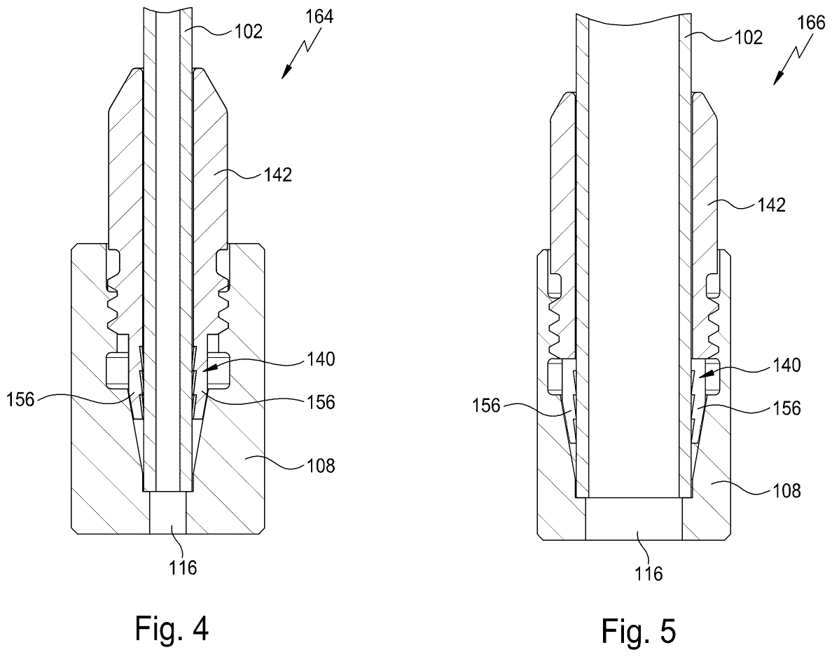

The pharmaceutical connection apparatuses 164 and 166 shown in part in FIGS. 4 and 5 are functionally identical to the pharmaceutical connection apparatus 100, but adapted to hose lines 102 of different sizes. While the hose line 102 has a smaller diameter in the variant in accordance with FIG. 4 than in the pharmaceutical connection apparatus 100, the hose line 102 in the variant in accordance with FIG. 5 has a larger diameter. Otherwise, what has been said above applies.

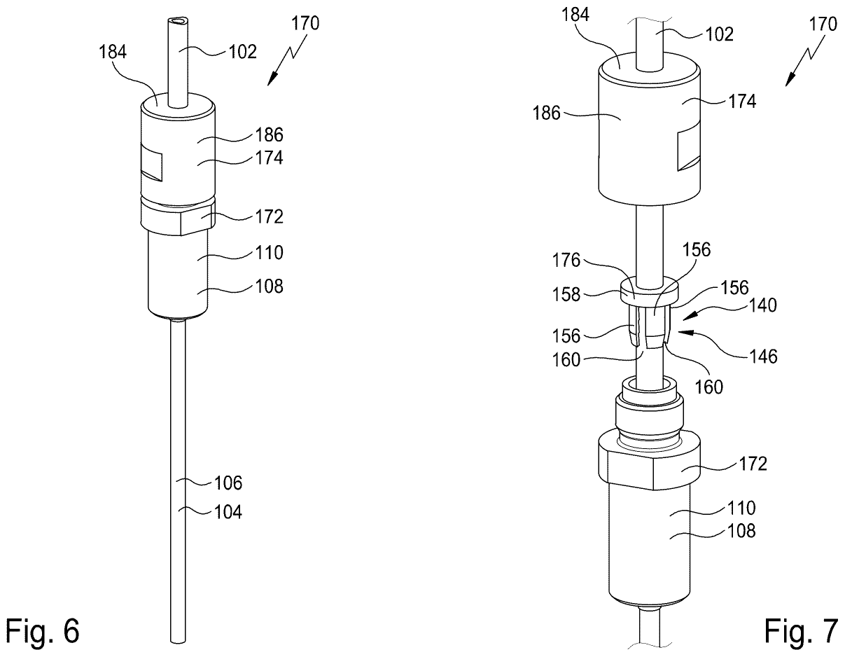

Reference numeral 170 designates an advantageous embodiment of the pharmaceutical connection apparatus, which is shown in FIG. 6 to 8.

In the pharmaceutical connection apparatus 170, in particular the clamping body 140 and the fixing device 136 are configured differently from the pharmaceutical connection apparatus 100.

In a manner deviating from the pharmaceutical connection apparatus 100, the base body 138 does not have a second portion 112 with receiving space 132. Adjacent to the first portion 110, in the present example a mounting portion 172 is provided, for example configured in a flange-like manner. Furthermore, a second portion 112 is provided, which has an external thread as a fixing element 134.

The clamping body 140 is not intended for direct attachment to the base body 108 in the case of the pharmaceutical connection apparatus 170. Instead, the fixing device 136 comprises a fixing body 174.

The clamping body 140 comprises, as a connection region 158, an in the present case annular collar 176 from which the clamping elements 156 protrude. The clamping elements 156 are pressed together by the clamping receptacle 128 as explained above, thereby fixing the hose line 102. Furthermore, a force is applied in the axial direction for improved sealing on the contact region 118.

A cylindrical portion 178 is formed in the base body 108 between the contact region 118 and the taper 130 of the clamping receptacle 128. Said cylindrical portion is dimensioned such that the hose line 102 is surrounded in a positive-locking manner. The portion 178 serves to stabilize and guide the hose line 102 at the end portion 126.

In order to fix the clamping body 140 in the usage state, the fixing body 174 of the fixing device 136 is provided with a fixing element 180. In the present case, said fixing element is configured as a screw element with an internal thread of the fixing body 174, which is configured in a cap-like shape, for example as a cap nut.

The hose line 102 passes through a through-opening 182 on an upper portion 184 of the fixing body 174, from which an edge 186, which carries the internal thread, protrudes in the direction of the base body 108.

In this way, the fixing body 174 overlaps the clamping body 140 and, in portions, the base body 108. The fixing elements 134 and 180 are screwed together.

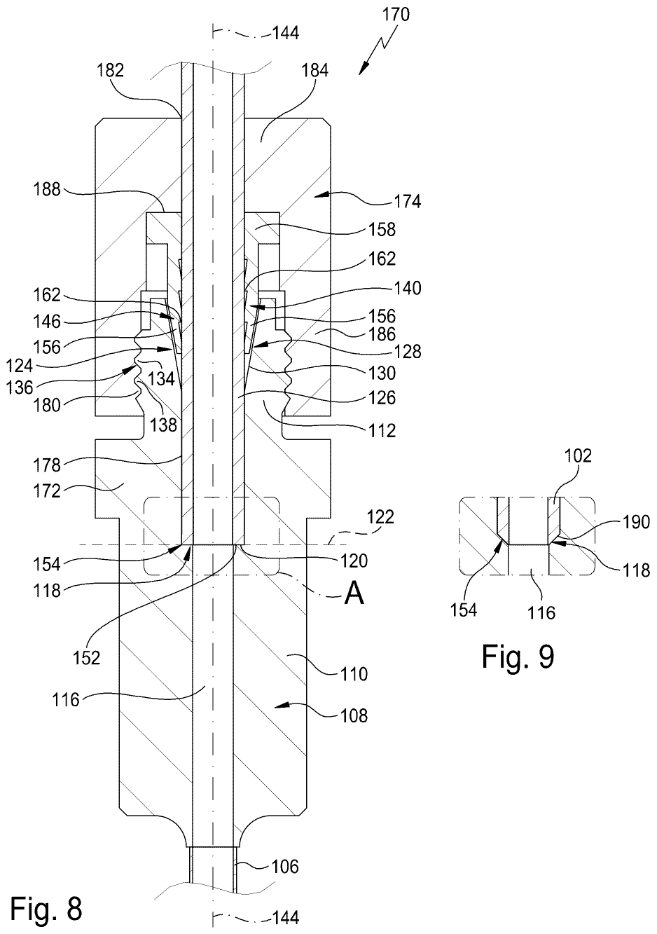

The clamping body 140 is supported, in the present case directly, on the fixing body 174 with a side 188 facing away from the base body 108. In particular, the fixing body 174 and the clamping body 140 preferably rest flat against each other via corresponding contact elements (FIG. 8).

Depending on how tightly the fixing body 174 and the base body 108 are screwed together, the hose line 102 is subjected to more or less force via the clamping region 146. The same applies to the force acting on the end face 152 at the sealing seat 154.

FIG. 9 is a detail (A) of FIG. 8 in a variant of the embodiment of the contact region 118. Such an embodiment can be used not only in the pharmaceutical connection apparatus 170, but, in a manner deviating from the respective illustration, can be provided for all The pharmaceutical connection apparatuses described here.

In the variant in accordance with FIG. 9, the contact region 118 is shaped in such a way that it is formed as a conical taper 190 of the base body 108 in the direction of the channel 116. The hose line 102, arranged in abutment, is thereby compressed. This can be advantageous for a better seal at the sealing seat 154.

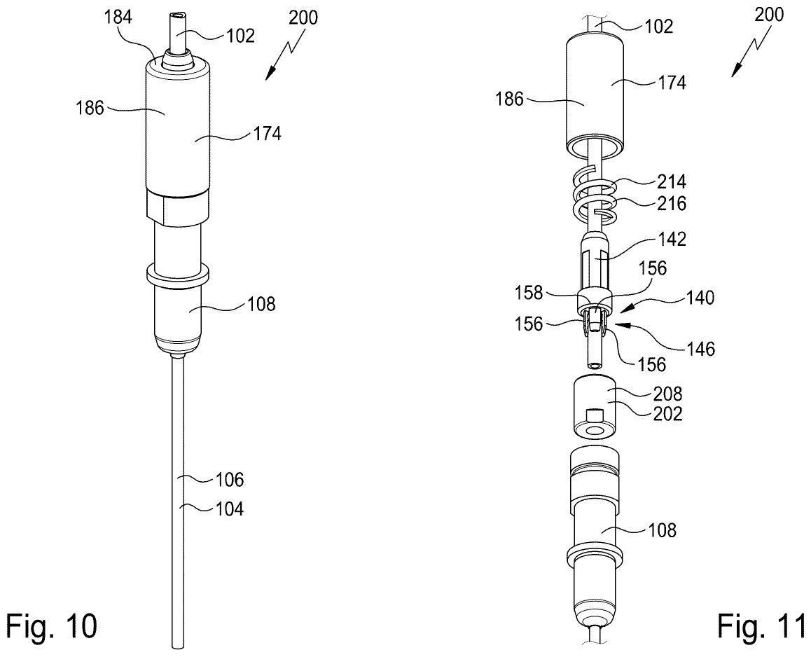

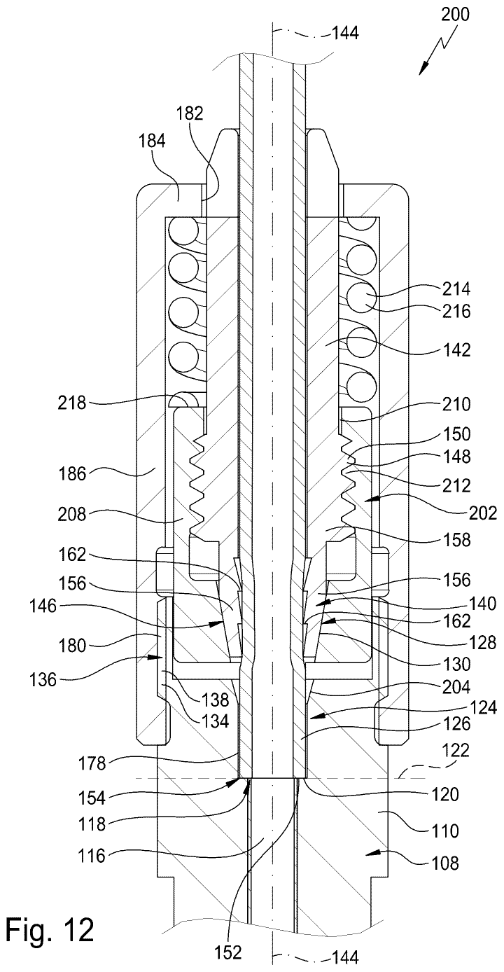

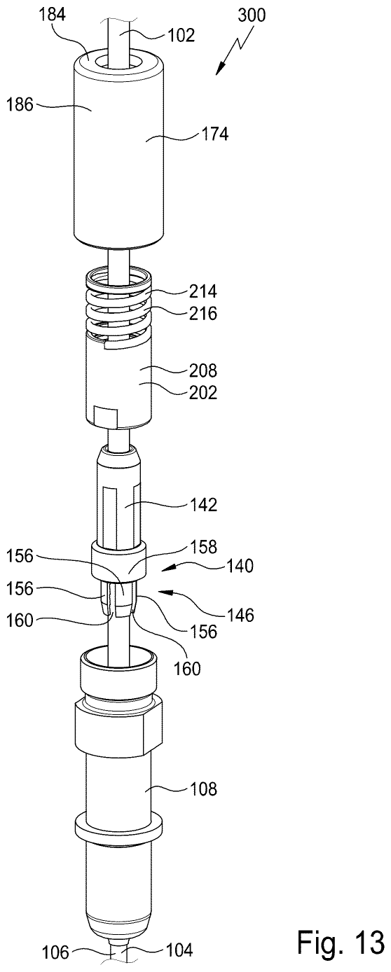

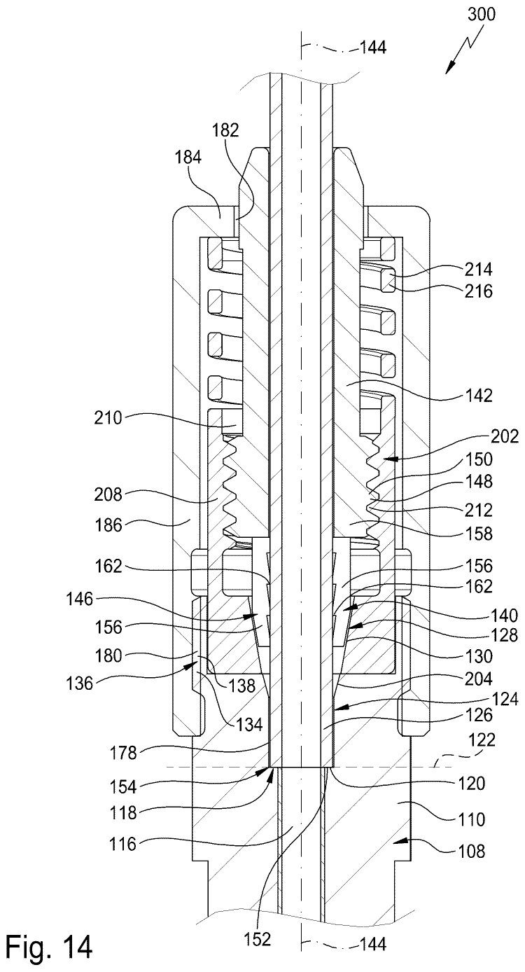

Further preferred embodiments are described below, with reference numeral 200 in FIG. 10 to 12 and reference numeral 300 in FIGS. 13 and 14. Regarding the embodiment, and from a functional perspective, there are many similarities between the pharmaceutical connection apparatuses 200 and 300, which can therefore be explained together first. The differences will then be discussed.

In the pharmaceutical connection apparatuses 100, 164, 166 and 170, the clamping receptacle 128 is formed with the taper 130 from the base body 108. The pharmaceutical connection apparatuses 200, 300, in which a receiving body 202 is used in each case to form the clamping receptacle 128, differ from this.

The base body 108 is substantially cylindrical and forms the channel 116 and the insertion region 124 located in front of it. In the present example, the portion 178 for surrounding the hose line 102 in a positive-locking manner is arranged at the insertion region. On the entry side at the insertion region 124, a taper 204, even if slight, is provided.

On the entry side, the receiving space 132 is formed on the base body 108, which space is enclosed by an edge 206 comprising the fixing element 134 in the form of the external thread.

For fixing the clamping body 140 by means of the fixing device 136, the fixing body 174 is also provided in the case of the pharmaceutical connection apparatuses 200. This comprises the fixing element 180, configured as an internal thread, for cooperation with the fixing element 134.

In the present case, the edge 186 is elongate, such that the fixing body 174 has an overall cylindrical shape with a longitudinal extent that considerably exceeds the diameter. However, this is only one embodiment by way of example.

The through-opening 182 is also provided, not only the hose line 102 but also the clamping body 140 passing through the through-opening 182.

The clamping body 140 has a shape that largely corresponds to the shape of the clamping body 140 in the case of the pharmaceutical connection apparatus 100. In the case of the pharmaceutical connection apparatuses 200, 300, the clamping body 140 is also configured as an elongate sleeve 142.

The clamping body 140 comprises the fixing element 148, configured as a screw element with external thread, and the clamping region 146 with a plurality of clamping elements 156.

The clamping receptacle 128 is formed by the receiving body 202. In the present case, the receiving body 202 is configured as a sleeve 208 that surrounds the clamping body 140. In the present example, the sleeve 208 has a shorter longitudinal extent (approximately half) in relation to the sleeve 142, but this is only by way of example.

The sleeve 208 comprises a through-opening 210 into which the clamping body 140 engages from the side facing away from the base body 108. The clamping receptacle 128 with the taper 130 on the sleeve 208 is formed facing the base body 108.

The sleeve 208 also comprises a fixing element 212, in the present case configured as an internal thread. The fixing element 212 is arranged on the side facing away from the base body 108.

The fixing elements 148 and 212 can cooperate in order to connect the clamping body 140 to the receiving body 202 by screwing. In this case too, the depth of the screw connection can influence the force applied to the hose line 102 on the clamping region 146 and the force with which the end face 152 rests on the sealing seat 154.

In the case of the pharmaceutical connection apparatus 200, 300, the clamping body 140 is not directly supplied with a force by the fixing body 174, unlike in the pharmaceutical connection apparatus 170. Instead, an indirect force is applied.

In the present example, a biasing element 214 is provided in each case, which is effective between the fixing body 174 and the receiving body 202. The biasing element 214 is configured as a spring element 216, in particular as a coil spring, that acts under pressure.

The spring element 216 rests against the fixing body 174, in particular on the upper portion 184, and can be supported against it. The opposite end of the spring element 216 rests against the receiving body 202, in particular against its end-face portion 218, which faces the upper portion 184.

Since the clamping body 140 is connected to the receiving body 202, the clamping body is indirectly supplied with the biasing force of the spring element 216. In the usage state, the clamping body 140 is thereby supplied with the force directed on the base body 108, for fixing the hose line 102.

The spring element 216 is arranged radially on the inside with respect to the fixing body 174 and is surrounded by its edge 206. With respect to the clamping body 140, the spring element 216 is arranged radially on the outside and surrounds it.

In the pharmaceutical connection apparatus 200, the spring element 216 is a separate component, formed separately from the receiving body 202 and separately from the clamping body 140.

In contrast, the biasing element 214 in the pharmaceutical connection apparatus 300 is integrally formed with the receiving body 202. Alternatively, a one-piece design using the clamping body 140 is conceivable, for example.

The one-piece (monolithic) production of the receiving body 202 and the biasing element 214 is advantageous from a manufacturing technology perspective. In this case, in particular an additive manufacturing method, such as 3D printing, is used.

In particular, the pharmaceutical connection apparatuses 200 and 300 are advantageously suited for cleaning and sterilizing the hose line without disassembly in a CIP/SIP process. In this case, hot steam can flow through the hose line 102, which steam is, for example, at a temperature of approximately 135° C. and a pressure of approximately 2 bar and above.

The biasing element 214 makes it possible to particularly reliably ensure that the hose line 102 does not lift off the sealing seat 154 and/or deform, even at high temperatures and high pressures, such that the advantageous sealing properties are maintained even in the case of long-term use and repeated sterilization.

### LIST OF REFERENCE SIGNS

100 The pharmaceutical connection apparatus
102 hose line
104 object
106 filling needle
108 base body
110 first portion
112 second portion
114 axis
116 channel
118 contact region
120 shoulder
122 contact plane
124 insertion region
126 end portion
128 clamping receptacle
130 taper
132 receiving space
134 fixing element
136 fixing device
138 screw element
140 clamping body
142 sleeve
144 axis
146 clamping region
148 fixing element
150 screw element
152 end face
154 sealing seat
156 clamping element
158 connection region
160 gap
162 projection
164 The pharmaceutical connection apparatus
166 The pharmaceutical connection apparatus
170 The pharmaceutical connection apparatus
172 mounting portion
174 fixing body
176 collar
178 cylindrical portion
180 fixing element
182 through-opening
184 upper portion
186 edge
188 side
190 conical taper
200 The pharmaceutical connection apparatus
202 receiving body
204 taper
206 edge
208 sleeve
210 through-opening
212 fixing element
214 biasing element
216 spring element
218 portion
300 The pharmaceutical connection apparatus

1. A pharmaceutical connection apparatus for a hose line, the pharmaceutical connection apparatus comprising:
a base body, in which an insertion region for the hose line and a channel are formed, wherein the insertion region opens into the channel and on an entry side of the channel a contact region for the hose line is arranged;
a clamping body, through which the hose line is guided and which comprises or forms a clamping region facing the base body;
a clamping receptacle, which has a taper in a direction of the contact region; and
a fixing device for fixing the clamping body relative to the base body in a usage state of the pharmaceutical connection apparatus;
wherein in the usage state, the clamping body engages, by means of the clamping region, into the clamping receptacle, and the clamping region applies a clamping force to the hose line radially from outside, and wherein the hose line extends by means of an end portion through the insertion region and rests with an end face on the contact region, which forms a sealing seat.

2. The pharmaceutical connection apparatus according to claim 1,
wherein the hose line is free from interference by the base body, wherein the hose line is not slipped over the base body.

3. The pharmaceutical connection apparatus according to claim 1,
wherein the base body forms the clamping receptacle, such that the clamping region is held in a clamping fit on the base body in the usage state for applying the clamping force to the hose line.

4. The pharmaceutical connection apparatus according to claim 3,
wherein the fixing device comprises cooperating fixing elements on the clamping body and on the base body, the clamping body being directly connected to the base body.

5. The pharmaceutical connection apparatus according to claim 4,
wherein the fixing elements are or comprise at least one of screw elements, clamping elements and locking elements.

6. The pharmaceutical connection apparatus according to claim 5,
wherein the clamping body engaging in the base body and an external thread of the clamping body being screwed to an internal thread of the base body, or the clamping body overlapping the base body and an internal thread of the clamping body being screwed to an external thread of the base body.

7. The pharmaceutical connection apparatus according to claim 1,
wherein the fixing device comprises a fixing body formed separately from the clamping body, which is connected to the base body via fixing elements, wherein the clamping body is directly or indirectly supported on the fixing body with a side facing away from the base body.

8. The pharmaceutical connection apparatus according to claim 7,
wherein the fixing elements are or comprise at least one of screw elements, clamping elements and locking elements, wherein the fixing body overlaps the base body and an internal thread of the fixing body is screwed to an external thread of the base body.

9. The pharmaceutical connection apparatus according to claim 1,
further comprising a receiving body which is configured separately from the base body and which forms the clamping receptacle, wherein the receiving body and the base body bearing against each other over mutually facing end faces, in the usage state of the pharmaceutical connection apparatus.

10. The pharmaceutical connection apparatus according to claim 9,
wherein the receiving body and the clamping body are connected to each other, and/or wherein the receiving body is configured as a sleeve that surrounds the clamping body at least in portions.

11. The pharmaceutical connection apparatus according to claim 10,
wherein the receiving body and the clamping body are connected to each other by at least one of screwing, clamping and locking, wherein an internal thread of the receiving body being screwed to an external thread of the clamping body.

12. The pharmaceutical connection apparatus according to claim 9,
wherein the fixing device comprises a fixing body formed separately from the receiving body and/or from the clamping body, which is connected to the base body via fixing elements, wherein at least one of the receiving body and the clamping body is directly or indirectly supported on the fixing body with a side facing away from the base body.

13. The pharmaceutical connection apparatus according to claim 12,
wherein the fixing elements are or comprise at least one of screw elements, clamping elements and locking elements, wherein the fixing body overlaps the receiving body and the base body, and an internal thread of the fixing body is screwed to an external thread of the base body.

14. The pharmaceutical connection apparatus according to claim 12,
further comprising a biasing element which is effective between the receiving body and/or the clamping body and the fixing body, the biasing element applying a force to the receiving body and/or the clamping body directed towards the contact region.

15. The pharmaceutical connection apparatus according to claim 14,
wherein the biasing element is configured as a spring element.

16. The pharmaceutical connection apparatus according to claim 14,
wherein the biasing element is integrally formed with the receiving body or integrally formed with the clamping body and/or is manufactured by an additive manufacturing process.

17. The pharmaceutical connection apparatus according to claim 14,
wherein the biasing element is arranged radially on an inside with respect to the fixing body, and/or wherein the biasing element surrounds at least one of the clamping body and the receiving body.

18. The pharmaceutical connection apparatus according to claim 1,
wherein the clamping body is configured as an elongate sleeve.

19. The pharmaceutical connection apparatus according to claim 1,
wherein the clamping body comprises two or more clamping elements on the clamping region, the two or more clamping elements being elastically deformable and separated from each other by at least one respective gap and held at a common connection region.

20. The pharmaceutical connection apparatus according to claim 19,
wherein the two or more clamping elements comprise more than two clamping elements, and the at least one respective gap comprises more than two respective gaps.

21. The pharmaceutical connection apparatus according to claim 19,
wherein the two or more clamping elements comprise two clamping elements that are opposite each other, and/or wherein the at least one respective gap comprises two gaps that are opposite each other.

22. The pharmaceutical connection apparatus according to claim 19,
wherein the two or more clamping elements comprise radially inner projections, and the two or more clamping elements facing the hose line have a sawtooth shape in cross-section.

23. The pharmaceutical connection apparatus according to claim 1,
wherein the base body is configured to be tapered at least in portions at the insertion region.

24. The pharmaceutical connection apparatus according to claim 1,
wherein one of the following applies:
the contact region defines a contact plane oriented perpendicularly to the channel;
the contact region is configured as a conical taper of the base body in a direction of the channel;
the contact region is located at an end of the taper of the clamping region, or wherein a cylindrical portion of the insertion region is formed on the base body.

25. The pharmaceutical connection apparatus according to claim 1,
wherein the base body comprises or forms a pharmaceutical filling needle for filling pharmaceutical containers, or wherein the pharmaceutical connection apparatus comprises a filling needle connected or connectible to the base body.
